(19) United States
(12) Patent Application Publication
(10) Pub. No.: US 2025/0040937 A1
(43) Pub. Date: Feb. 6, 2025

(54) WEARABLE GARMENT WITH BLOOD DETECTION AND AUTOMATED COMPRESSION ASSEMBLY

(71) Applicant: 0D Technologies, LLC, Aventura, FL (US)

(72) Inventor: David M. Sharvit, Aventura, FL (US)

(73) Assignee: 0D Technologies, LLC, Aventura, FL (US)

(21) Appl. No.: 18/793,095

(22) Filed: Aug. 2, 2024

Related U.S. Application Data
(60) Provisional application No. 63/517,762, filed on Aug. 4, 2023.

(51) Int. Cl. A61B 17/132; A61B 17/00; H04W 4/90

(52) U.S. Cl. CPC A61B 17/1322; A61B 2017/00022; A61B 2017/00039; A61B 2017/00398; H04W 4/90

(57) ABSTRACT

A wearable garment that is configured to detect the presence of a hemorrhage or bleeding and automatically activate a torniquet assembly. The wearable garment comprises a detection assembly, a torniquet assembly and/or a micro-controller and/or micro-processor, all disposed within various layers thereof. The detection assembly comprises sensors configured to ascertain a positive reading, i.e., the presence of blood in the wearable garment. The processing assembly is configured to activate the torniquet assembly upon a positive blood reading and/or to send an EMS signal to first responders. The torniquet assembly comprises a wiring system disposed around various sections of the sleeves of the wearable garment. The wiring system is coupled to a modified spool and operatively connected motor, which is configured with the micro-controller to automatically operate an ascertained blood reading, which may be indicative of a hemorrhage and/or bleeding around a body artery.

### FIELD OF INVENTION

The present invention relates to wearable garments and clothing items with a blood detection and automated compression assembly.

### BACKGROUND

Uncontrolled hemorrhages, including from injured limbs, are currently the leading cause of death as it relates to combat casualty care and are the second highest cause of traumatic death in the civilian sector. Wounds from sharp instruments, such as knifes, glass, etc., continue to represent a significant factor in causing bleeding and/or hemorrhages. Uncontrolled bleeding and hemorrhages pose a medium risk of an individual becoming unconscious and a high risk of losing a limb and even bleeding out before medical assistance may be provided. However, the response time associated with attending a bleeding emergency is generally high when compared to the small timeframe to successfully treat a wounded individual. While there are some products that may protect an individual from such wounds, e.g., bullet proof vests armored wear, etc., such existing products are not perfect and also fail to provide any assistance to a wounded person. Said differently, their protection is limited to acting merely as a shield that does not prevent all combat or civilian related wounds. In addition, existing products are often times bulky, heavy and may not be integrated easily or function as clothing or even a fashion item. Furthermore, given such small windows involved, i.e., to prevent loss of limb or life after a wound, it becomes crucial to effectively and rapidly seek medical assistance in the event of a serious wound. Sometimes, even in urban centers, it is nearly impossible to provide such effective and rapid assistance given the delays that are generally associated in contacting and informing first responders after an emergency.

Given the foregoing risks of loss of limb and the lack of practical alternatives to aid in the event of serious wound to an individual, the relevant industry would greatly benefit from providing a wearable garment that could automatically apply a torniquet system in the event of a serious wound. An important benefit would be realized in the industry by providing a wearable garment with a motorized wire or lacing system operatively connected to a group of sensors that could automatically ascertain the presence of blood once detected. A further benefit would be realized if such wearable garment could be configured with small electronic components, including a micro-controller and/or a microprocessor, to ascertain a blood reading and automatically generate compression on the affected limb(s) be reducing the length of the wire or lacing system. An even further benefit would be realized if such electronic components could be configured with the sensors to automatically send an alert to first responders upon a successful detection of blood. Yet a further benefit would be provided if such a wearable garment could function as a clothing or even a fashion item seamlessly integrating and concealing its internal components.

### SUMMARY

The present invention is directed to a wearable garment that is configured to detect the presence of a hemorrhage or bleeding and automatically activate a compression assembly. The wearable garment comprises a top section and/or a bottom section, each one having a first or inner layer, a mid-layer and an upper layer. The wearable garment also comprises detection assembly disposed between the inner layer and mid-layer or between the mid-layer and outer layer, and a compression assembly disposed between the mid layer and the outer layer. The wearable garment also comprises a microprocessor(s) and/or a microcontroller(s) operatively connected to a battery system for power and disposed in a specialized case, e.g., coated with Teflon® and/or configured and dimensioned to be disposed within the plurality of front pockets. Alternatively the microprocessor(s) and/or microcontroller(s) may be disposed within the mid-layer and the outer or alternatively around one or more of a plurality of pockets or pouches disposed on the exterior of the outer layer.

The compression assembly comprises a compression structure disposed around sleeves of the top section and/or bottom section, generally at intended areas proximate to the location of the main arteries of the user. The compression structure, which is disposed between the mid-layer and outer layer, is operatively connected to a motorized spool that is operatively connected to the compression structure and configured to rotate clockwise and counter-clockwise, e.g., due to the driving force of a motor located above the outer layer or a designated pouch as described herein, e.g., disposed on the exterior of the outer layer. A clockward rotation of the spool is generally intended to wind the compression structure upon the stool, which consequently results in a compression of the compression structure on the individual, e.g., on an arm or leg. Conversely, a counter- clockwise rotation of the spool causes a decompression of the wiring. The detection assembly comprises a plurality of sensors operatively disposed within the wearable garment at the intended areas or in proximity thereto. The sensors are operatively configured to automatically activate the motor upon a successful detection of certain data characteristics associated with blood around the intended area(s) of compression. As used herein, a successful detection of certain data characteristics indicative of the presence of blood within at least one of the various layers of the wearable garment will be referred to as a “positive reading.”

The wearable garment may also comprise a microcontroller(s) and a microprocessor(s), including with communication unit. The microcontroller(s) and/or the microprocessor(s) may be operatively disposed with the detection assembly and the compression assembly to enable the automatic operation of the motor/spool and/or to send an emergency signal upon a positive reading.

### BRIEF DESCRIPTION OF THE DRAWINGS

FIG. 1A is a front view of the wearable garment according to the present invention.

FIG. 1B is a side view of the wearable garment according to the present invention.

FIG. 1C is a front view of a user showing the approximate location of the body's main arteries.

FIG. 2 is a perspective view of a portion of one embodiment of the mid-layer of the wearable garment according to the present invention.

FIG. 3A is a perspective view of a portion of one embodiment of the mid-layer of the wearable garment according to the present invention.

FIG. 3B is a front view of a portion of one embodiment of a layer of the wearable garment according to the present invention comprising sleeve openings.

FIG. 3C is a perspective view of one embodiment of a portion of the wearable garment according to the present invention.

FIG. 3D is a diagrammatic representation of one embodiment of the layer arrangement according to the wearable garment of the present invention.

FIG. 4 is a back view of a portion of one embodiment of the wearable garment according to the present invention.

FIG. 5A is a top view of a portion of one embodiment of a sleeve of the wearable garment according to the present invention.

FIG. 5B is a is a diagrammatic representation of one embodiment of the processing assembly according to the present invention.

FIG. 5C is a front and side view of other embodiments of the wearable garment according to the present invention.

FIG. 6A is a top view of one embodiment of a motor of the torniquet assembly according to the present invention.

FIG. 6B is a perspective view of various embodiments of the motorized components of the torniquet assembly according to the present invention.

FIG. 7 is a perspective view of a portion of one embodiment of the processing assembly according to the wearable garment of present invention.

### DETAILED DESCRIPTION

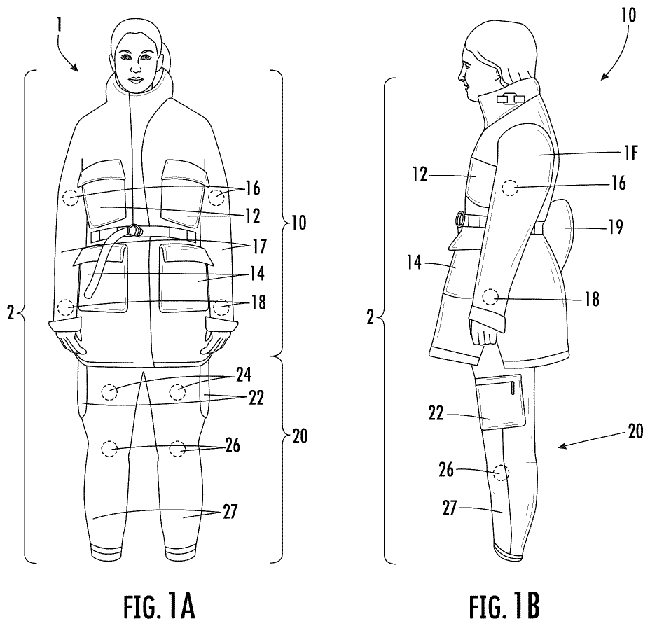

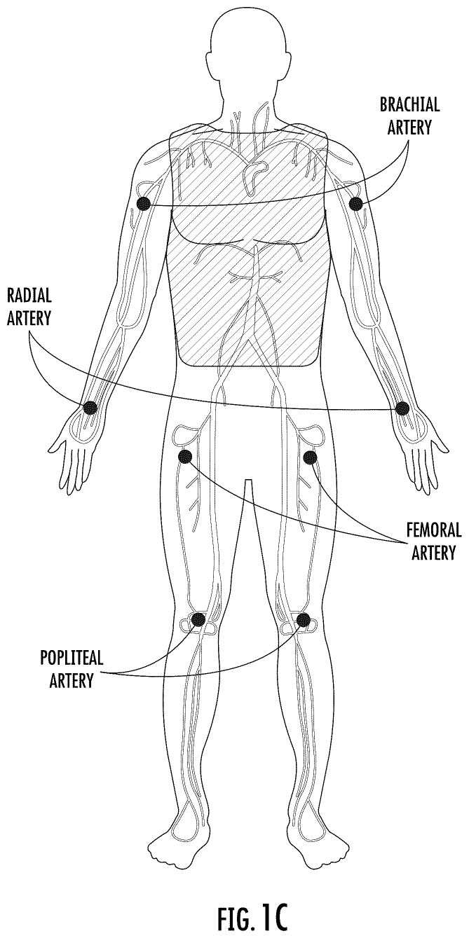

With initial reference to at FIGS. 1A-7, the present invention is directed to a wearable garment with an automated torniquet system, which for simplicity will be herein referred to as wearable garment assembly 1. The inventive wearable garment is intended to automatically activate compression mechanism upon the detection of blood within the interior wearable garment assembly 1, for example, in the event that the user starts to bleed from a wound, e.g., gunshot wound, stabbing wound, etc. Accordingly, the wearable garment assembly 1 according to the present invention comprises multi-layer assembly 2 comprising at least a layered top section 10, e.g., jacket, and/or optionally a bottom section 20, e.g., pants. The wearable garment assembly 1 also comprises a compression assembly 30, a detection assembly 40, a microprocessor 50, and/or a microcontroller 59 disposed on the wearable garment assembly 1.

Accordingly, the detection assembly 40 and the microcontroller 59 are generally configured with one another to dispose the compression assembly 30 in an “operative orientation” upon detecting a predetermined change in at least data characteristic, for example, a voltage value, or a change in voltage, associated with the presence of the user's blood in a targeted location of the wearable garment assembly 1. As used herein, the operative orientation is associated with an increase in the pressure or compression force exerted on the individual by the compression assembly 30. The detection assembly 40 and the microcontroller 59 are also generally configured with one another to dispose and/or maintain the compression assembly 30 in an “inoperative orientation” upon detecting a voltage value, or a change in voltage, that is not associated with the presence of blood. As used herein, the inoperative orientation is generally associated with an at least partial decrease in the pressure exerted on the individual by the compression assembly 30. In addition, the detection assembly 40 and the microcontroller 59 are also generally configured with one another to automatically dispose and/or maintain the compression assembly 30 in an inoperative orientation after a predetermined period that it has been disposed in the operative orientation. By ways of example only, the compression assembly may be disposed in the operative orientation for about fifteen (15) minutes to activate a compression cycle. Thereafter, the compression assembly 30 may be automatically disposed in the inoperative orientation for about two (2) minutes. This may be advantageous to prevent permanent damage to the body of the individual, e.g., at the limb(s), which may result by a prolonged pressure applied by the compression assembly 30. After a decompression cycle, i.e., when the compression assembly 30 is disposed in the inoperative orientation for about two minutes after an initial compression cycle, the compression assembly 30 may be once again disposed into the operative orientation to begin a second compression cycle. This process may be repeated as needed or until the patient receives medical attention. A controlled release of at least some of the pressure exerted by the compression assembly 30, i.e., when disposed in the inoperative orientation after a compression cycle, may be advantageous to at least partially allow some circulation, which may save the limb(s) of the user. For example, the microcontroller(s) and/or detection assembly 30 may be configured to reduce the pressure exerted by the compression assembly 30, e.g., for about 2 minutes for approximately every 15 minutes after it has been disposed in the operative orientation.

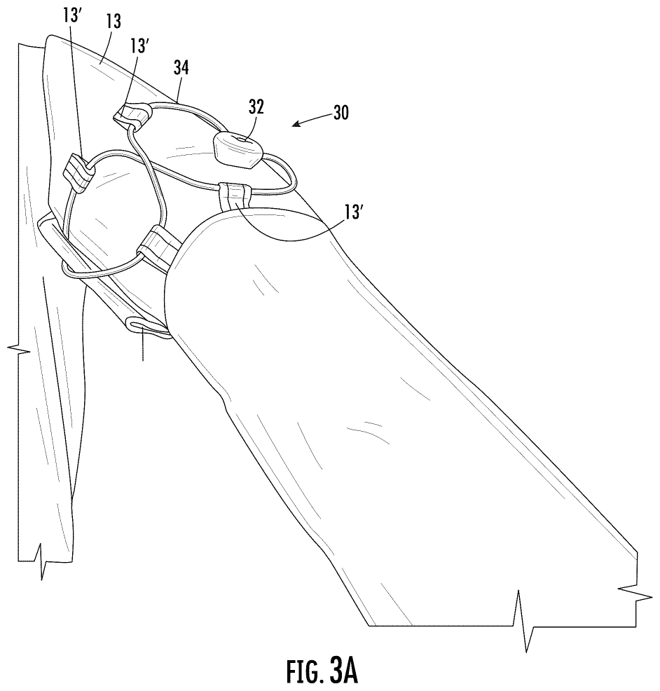

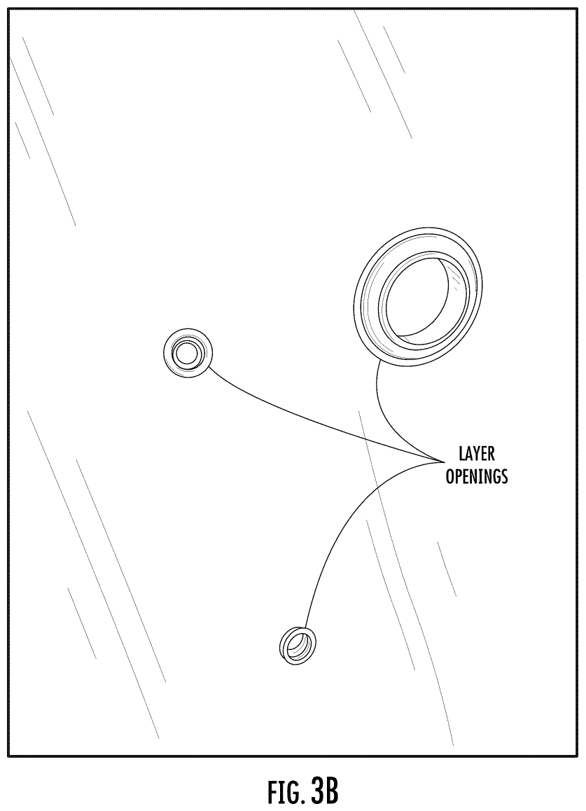

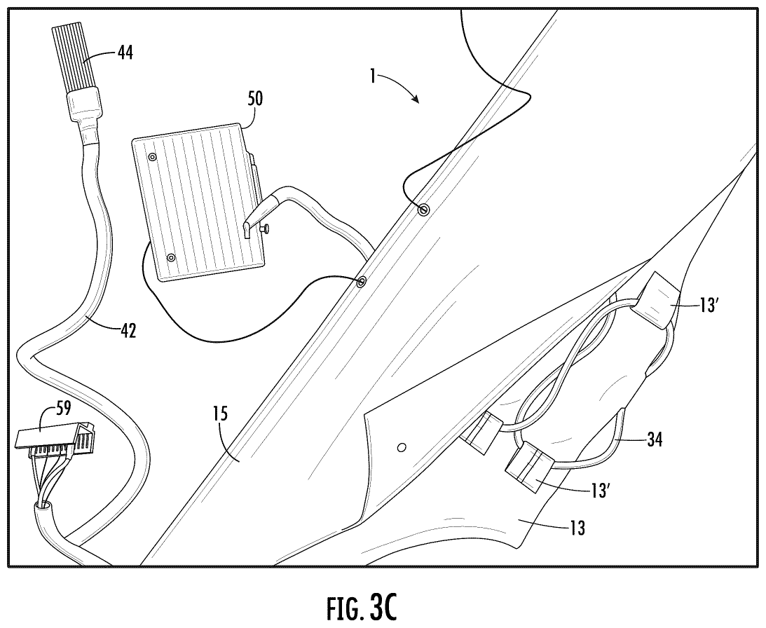

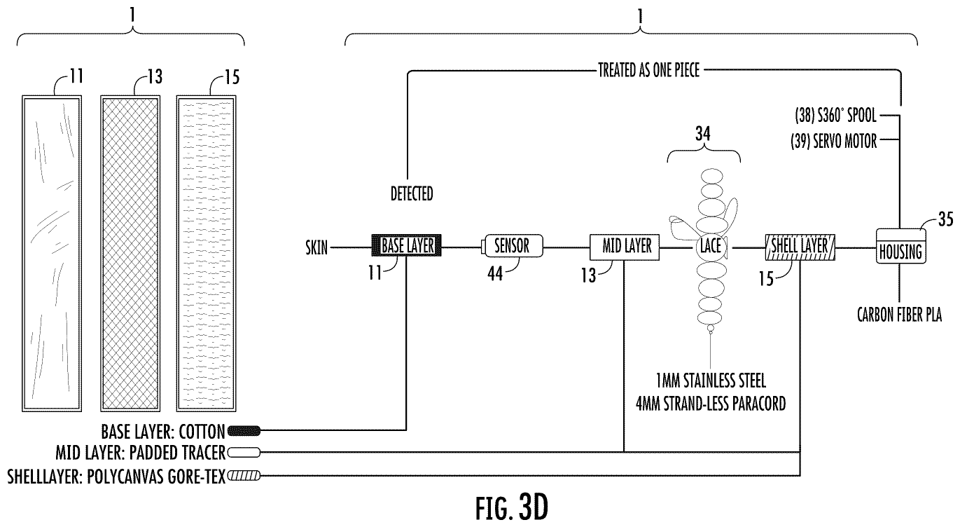

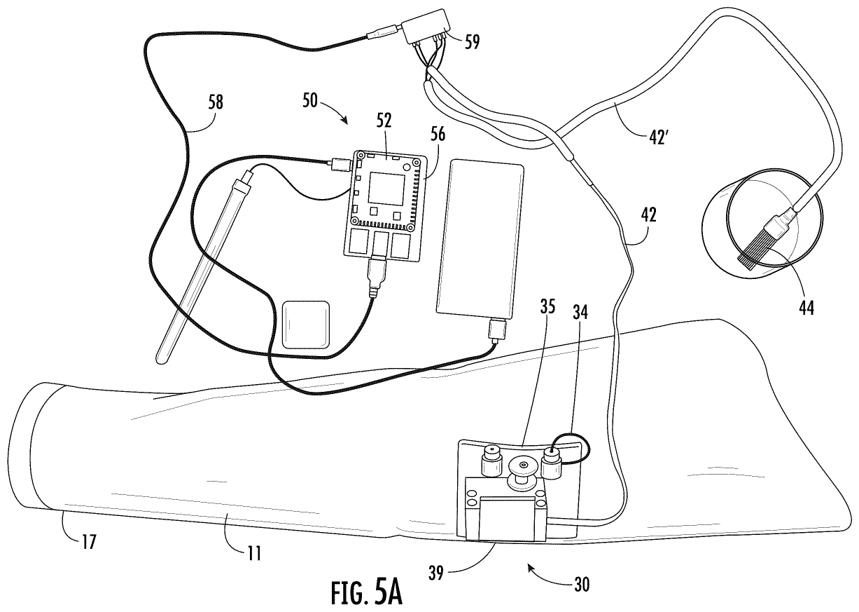

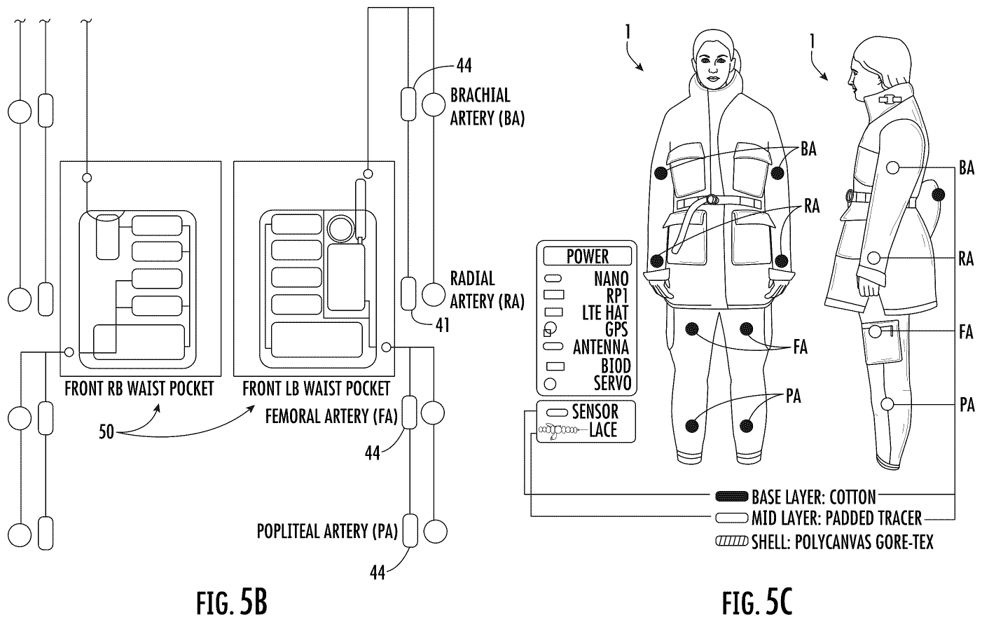

With reference to FIGS. 1A-1B and as briefly mentioned above, the wearable garment assembly 1 according to the present invention generally comprises a top section 10 or “jacket” with a plurality of pockets 12 and 14 that may be used to store items and/or house at least some of the operative components of the wearable garment, e.g. the microprocessor 50 and/or microcontroller 59 as described herein, and/or a power source for these components and/or the motor 38 such as a battery system. Other components, such as the compression assembly 30 and/or the detection assembly 40 may be disposed between the various layers. For example, the detection assembly 40 may be disposed between the inner 11/21 and mid layers 13/23, whereas the compression assembly 30 may be disposed between the mid 13/24 and outer 15/25 layers as defined herein. Additionally, the bottom section 20 or “pants” may also be provided with a plurality of side pockets 22 that may achieve a similar purpose. As is shown in FIG. 1B, the upper section 10 or “jacket” may comprise a centralized pouch unit 19, which may be used to store similar components in addition to the compression assembly 30, which is perhaps different than the illustrative embodiment of FIG. 1A whose motor 39 and spool 38 combination may be housed and/or disposed on the outer layer at 16, 18, 24 and/or 26. As is perhaps best represented in FIG. 5C, the top section 10 and/or bottom section 20 may comprise a plurality of layers, including an inner layer or base layer 11 and/or 21, a mid-layer 13 and/or 23 and an outer layer or shell layer 15 and/or 25. As an illustrative example, the inner or base layer 11 and/or 21 may comprise an absorbent material, such as highly absorbent cotton, as it will be in contact with an under garment and/or the skin of the user. As a further example, the mid layer 13 may comprise a similar or different material or combinations thereof and/or a padded fabric material. As an even further example, the outer or shell layer 15 and/or 25 may comprise a waterproof poly-canvas fabric. Accordingly, and with reference to at least FIG. 3D, which shows a visual representation of the inventive layered arrangement, it is contemplated that the wearable garment assembly 1 function as a unitary garment.

With reference to FIGS. 1A-1C, it is within the scope of the present invention that the compression assembly 30 should be able to apply or otherwise exert sufficient pressure to certain areas of the body of the user to control or otherwise at least partially reduce bleeding and/or hemorrhaging. For example, as represented in FIG. 1C, there are eight (8) areas in the human body where it may be advantageous to apply a torniquet to control bleeding and/or hemorrhaging. These areas shown in FIG. 1C are associated with the locations of the branchial artery (“BA”), the radial artery (“RA”), the femoral artery (“FA”) and the popliteal artery (“PA”). Accordingly, as shown in FIGS. 1A and 1C, these areas are represented on the top section 10 of the wearable garment assembly 1, e.g., on sleeves 17, as 16 for the BA and 18 for the RA. Likewise, these areas are represented on the bottom section, e.g., on sleeves 27, of the wearable garment assembly 1 as 22 for the FA and 26 for the PA.

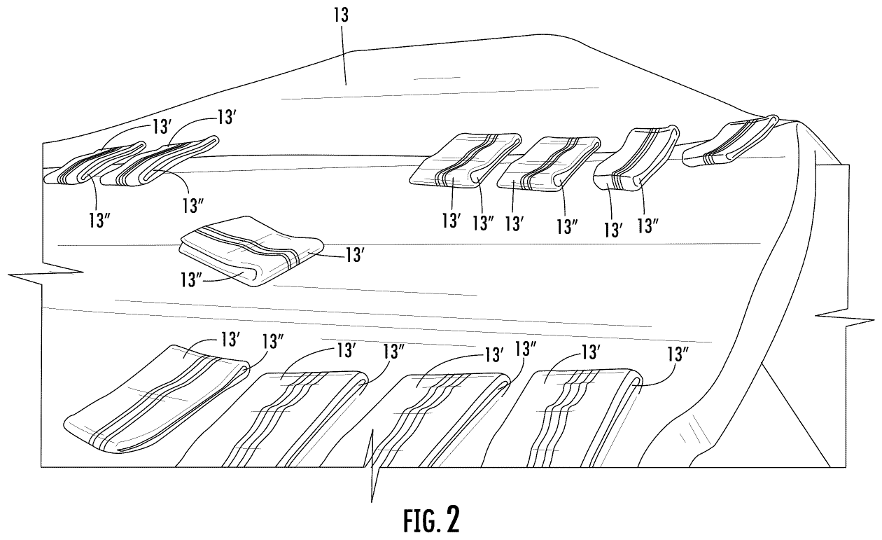

With reference now to at least FIGS. 3D, the components of the compression assembly 30 are generally disposed between the inner layer(s) 11 and/or 21 and the mid- layer(s) 13 and/or 23. As showing in FIG. 2, various interior sleeves 13′ may be disposed on an outer surface of the mid layer(s) 13/23, e.g., between the outer layers 15/25 and mid-layers 13/23. Accordingly, the interior sleeves 13′ should be disposed between the inner layer(s) 11 and/or 21 and the mid-layer(s) 13 and/or 23 in non-movable relation thereto. The interior sleeves 13′ generally comprise openings 13″ which may be configured and dimensioned to accommodate the size of a compression structure 34. For example, the compression structure may comprise a tensioning element such as a cable, wire, lacing, or other comparable structure that may pass through and may reciprocally move within the openings 13″ of the interior sleeves 13′. As shown at least in the illustrative embodiment of FIG. 3A, the compression structure 34 may pass through the sleeve openings 13″ around locations 16, 18, 24 and/or 26, including around a circumference of the sleeve(s) 17 and/or 27 to provide coverage to at least a portion or substantially all of the circumference of the limb, e.g., leg(s) or arm(s). In turn, the compression structure 34 may be configured to define braids, loops, or other geometrical patterns to provide for an increased coverage and/or effectiveness of the compression assembly 30. As such, and as may be best appreciated in FIG. 3A, it is contemplated that the compression structure 34 may reciprocally and/or slidably move through the opening of the interior sleeves 13′. As may be appreciated at least in FIG. 3C, if the compression structure 34 comprises steel wire, when compressed, it may pass through the inside of a gutless paracord for traction and continued cushion. As such, the compression structure 34 may be selectively contracted and/or tightened to dispose the compression assembly 30 in the operative orientation. Conversely, the compression structure 34 may also be selectively loosened to relieve pressure or otherwise dispose the torniquet 30 assembly into the inoperative orientation.

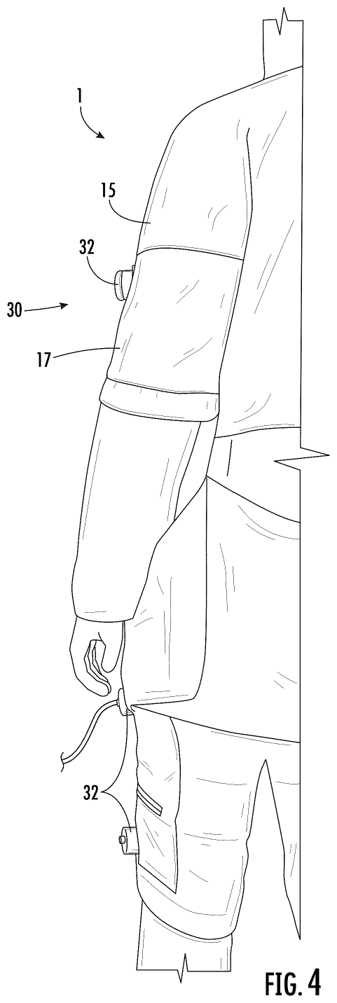

With reference now to FIG. 3A-4 and 5A, the compression structure 34 may be operatively connected to a spool 38 of compression assembly 30, which may rotate to reduce the length of the compression structure 34. As is perhaps best show in FIGS. 3 and 4, the or spool 38 may be disposed on either of the layers 11/21, 13/23, and/or 15/25, and may be operatively connected to the compression structure 34. By way of Example and with reference to at least FIG. 3B, the spool 38 may be operatively connected to the compression structure 34 through “layer openings”, e.g., to the pockets shown in FIG. 1A and or to the centralized pouch unit 19 shown in FIG. 1B.The spool 38 may comprise a cylinder that may rotate, e.g., via a motor 39 and spool 38, and as such, may coil the compression structure 34. When the compression assembly 30 is disposed in the operative orientation, the compression structure 34 should create a sufficient pressure around the circumference of the limb to at least partially, and in some cases fully, reduce blood flow along a corresponding artery, e.g., BA, RA, FA and/or PA. Examples of pressure ranges applied to a limb, which may be sufficient to at least partially reduce blood flow or hemorrhaging, may comprise about 50 millimeters of mercury (mmHg) to about 500 mmHg (which is about 69 grams per square centimeter (g/cm²) to about 680 g/cm²), with an average compression force of 130 mmHg (which is about 177 g/cm²). Other pressure ranges may comprise about 50 mmHg to about 70 mmHg (which is about 69 grams per square centimeter (g/cm²) to about 95.17 g/cm²) for injured arms, or about 75 mmHg to about 100 mmHg for injured legs (which is about 101.96 grams per square centimeter (g/cm²) to about 135.95 g/cm²). Accordingly, When the compression assembly 30 is disposed in the operative orientation, such reduction in the arterial blood flow should be sufficient to at least partially reduce the likelihood of death in the event of hemorrhaging and/or bleeding. Accordingly, the compression structure 34 may comprise a sufficient strength such that it does not snap or break when subjected to such pressure and/or stress. Furthermore, when the compression assembly 30 is disposed in the operative position, the spool 38 intended to rotate and coil the compression structure 34 to reduce their length along the circumference of the sleeve 17 and/or 27 to create pressure around the intended limb and to enable the operative orientation.

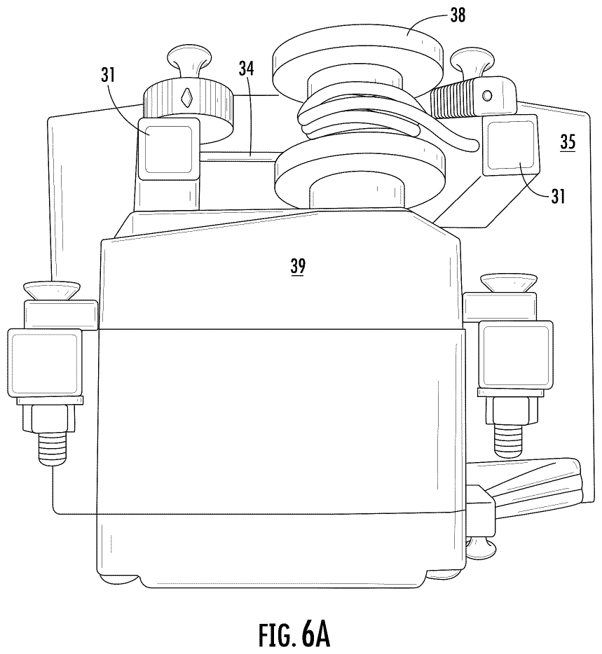

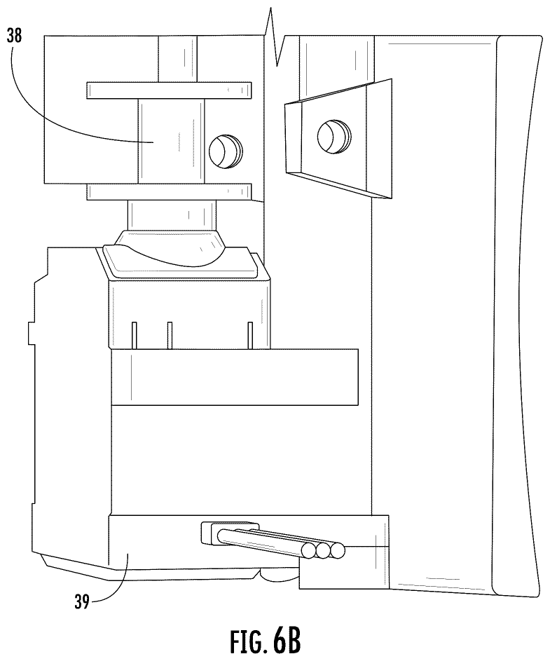

With reference to FIGS. 5A and 6A-6B, and as briefly mentioned above, the compression assembly 30 according to the present invention may comprise a motor 39, e.g., that has been mechanically adjusted or modified, that drives the rotation of the spool 38. As is contemplated herein a clockwise rotation of the motor 39 may be configured to tighten/compress the compression structure 34, whereas a counter-clockwise rotation of the motor 39 may be configured to loosen/decompress the compression structure 34. Such rotation may involve, for example, a 360 degree rotation of the spool 38. As a further example, as is perhaps best represented in FIG. 6A, the motor 39 may be mounted on a housing 35, with attendant mounting components thereof, and with sufficient clearance for the rotation and/or movement of the wire and/or lacing 38 around it. The housing 35 may be configured and dimensioned according to an intended size. As shown in FIG. 6A, the spool 38 may comprise one or more mounting guides 31 with openings disposed thereon to guide the compression structure 34 along the drum or coil of the spool 38. As an example, the housing 35 and/or mounting guides 31 with openings for wire passage may be 3D printed. As contemplated by the present invention, the material of the housing 35 and/or the centralized pouch unit 19 can comprise a Teflon® coating or a similar coating.

As may be also appreciated from a review of FIG. 6B, a stepper motor 39, i.e., no power to torque motor but operatively powered by a driver, may be operatively connected to the steel wire, and may be operatively configured to rotate and reduce or increase the length of the connected steel wire. As a further example, and as is also shown in FIG. 6B, a 25 kg motor operatively connected with a spool 38 may be provided, which may deliver a substantially continuous rotation or counter-rotation that may decrease or increase the length of the connected wire. The wire itself may comprise a sleeve or jacket to protect it and/or at least partially facilitate its movement. Alternatively, and as is perhaps best represented in FIG. 3C, a lacing material may be provided in lieu of steel wire. A lid may be provided as a cover to the housing 35 to cover the interior components thereof, including motor 39, spool 38, and/or compression structure 34. The lid may comprise various informational or decorative text, images, indicators, etc. Further, and with reference to the illustrative example represented in FIGS. 3B-3C, it is contemplated that the various layers 11/21, 13/23 and/or 15/25 may comprise various openings, including with reinforced sleeves to allow for passage of compression structure 34 from one layer to another, including in addition to any wires that may need to travel between the electrical components for the operation of the compression assembly 30 and/or detection assembly 40.

As shown at least in FIG. 5A, the wearable garment assembly 1 according to the present invention comprises a detection assembly 40. A plurality of sensors 44 of the detection assembly are generally disposed between the inner layer 11 and/or 21 and the mid-layer 13 and/or 23. However, this is not necessarily limiting, as the sensors 44 of the detection assembly 40 may also be disposed between the mid-layer 13 and/or 23 and the outer layer 15 and/or 25. The detection assembly 40 generally comprises a plurality of sensors 44 operatively connected to a microcontroller 59, a microprocessor 50 and/or the compression assembly 30. For example, various connections, e.g., 42, 42′ and/or 58 may be provided to operatively connect the sensors 44 of the detection assembly 40 and/or the compression structure 34 of the compression assembly to a microcontroller 59, a microprocessor 50 and/or to the motor 39.

The sensors 44 of the detection assembly 40 should be disposed on the wearable garment assembly 1 around an intended area(s), i.e., 16, 18, 24 and/or 26, or within sufficient proximity thereof and to the approximate location of a wound such that they may be in direct contact with blood in the event of bleeding/hemorrhaging. It is contemplated that the sensors 44 may be able to ascertain certain data characteristics associated with blood, including, without limitation a voltage associated with blood and/or other parameters such as conductivity, viscosity, density, constituents thereof, etc. Accordingly, the sensors 44 should be able to ascertain within a predetermined timeframe the presence of blood in around an intended area(s), i.e., 16, 18, 24 and/or 26, which is herein referred to as a positive reading.

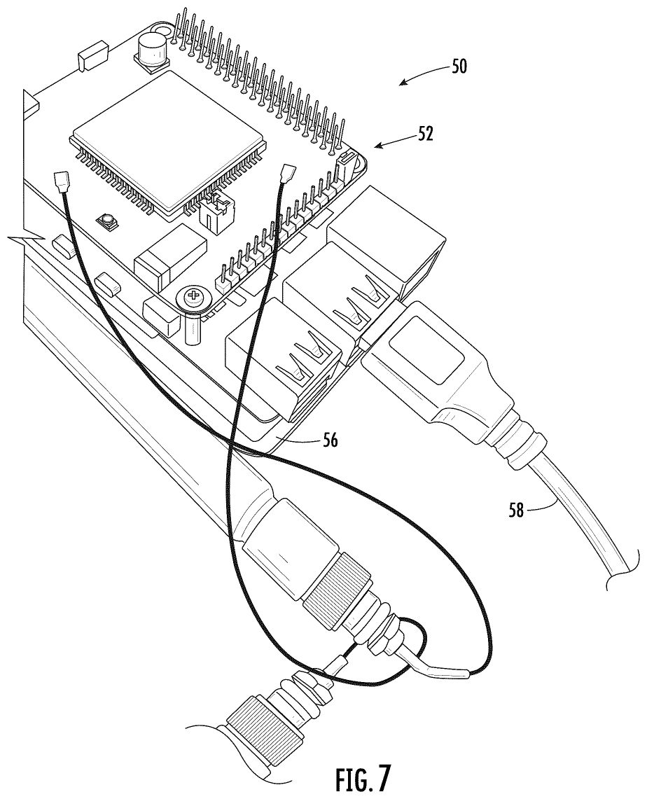

With reference now to FIGS. 5A-5C and 7, the wearable garment according to the present invention comprises a microcontroller 59 that may be configured to dispose the compression assembly 30 into the operative orientation upon an ascertained reading that is indicative of the presence of blood and/or bleeding/hemorrhaging. As such, the microcontroller 59 may obtain an ascertained data from the sensors 44 and may interpret an ascertained positive reading to control the operation of the motor 39 to enable the operative orientation. For example, the sensors 44 and/or microcontroller 59 may be manufactured by the company Audrino®, and may comprise pins that can measure the voltage of a substance, including a fluid, i.e., blood, sweat, moisture, etc. Such pins or analogue pins may be labeled with an “A” followed by a numeral, i.e., A0, A1, etc. Further to this example, such sensors 44 may be able to ascertain and/or interpret a voltage ranging from 0 to 5 volts and may have a function to convert such analogue voltage reading from a predetermined numeric value, e.g., ranging from 0 to 1023. Accordingly, the sensors 44 and/or microcontroller 59 may use a 10-bit analog-to-digital converter (ADC), such that it can represent such ascertained values using 1024 steps. In order to arrive at an actual voltage reading, the following formula may be used:

V=Voltage, X=Analogue Value

$$( X \div 1024 ) \times 5 = V$$

In the foregoing formula, the Numeric Value (X) is the ascertained analog reading and the integer 5.0 represents a default voltage reference, such as 5.0 volts. Further, the denominator 1024 is the number of steps that ADC can measure. The foregoing formula results in a voltage reading (in volts=V) based on an ascertained voltage reading.

It should be noted that two different sensor 44 units may not always produce and/or ascertain the same voltage given that small manufacturing differences, or sensor tolerances, can lead to variations in their output ranges. Such variations or differences between different sensors may be the result of component variations, sensor material variability, assembly process, etc. Due to these manufacturing differences, sensors 44 may require calibration to achieve consistent and accurate results according to the specific application. Factory calibration is a process where each device and/or sensor 44 is individually tested and the value ranges are adjusted to ensure it provides a consistent output. During calibration, the sensor's 44 output, i.e., the ascertain data, which may be transmitted to the microcontroller 59, is compared to a known reference or standard, and any deviations may be recorded and compensated.

Additional features of the present invention comprise providing a microprocessor 50 with a housing 56 to protect the interior components thereof. The microprocessor 50 may comprise a communication unit 52, which may comprise communicational and/or GPS locational and/or navigational capabilities. It is contemplated that upon a positive reading, the communication unit 52 be operatively configured with the microcontroller 59 to interpret the ascertained data, i.e., a positive reading, and automatically transmit a signal, e.g., an EMS signal, including via SMS, to the authorities, first responders, etc. The signal may comprise information associated the specific wearable garment assembly 1, e.g., name/last name of user, location within the garment/body of the bleeding, etc. The signal may also comprise information associated with the positive reading itself, e.g., physical location of the reading, time of reading, L. brachial, etc. In addition, upon a positive reading, the communication unit 52 may be configured to send a link to a GPS location of the user/wearable garment assembly 1 and an option to navigate to it, which may be accessed, for example, via a mobile device.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

What is claimed is:

1. A wearable garment assembly configured to exert a pressure on the body of a user upon a successful detection of blood within the garment assembly, the wearable garment assembly comprising:
a multi-layer assembly comprising:
a mid-layer disposed between an inner layer and an outer layer, and
a retention mechanism disposed between either the inner layer and the mid- layer or between the mid-layer and outer layer,
a compression assembly comprising a compression structure disposed within the retention mechanism in slidable relation thereto;
a detection assembly comprising a plurality of sensors each one disposed at an intended location of the inner layer, and
at least one microcontroller operatively connected to the detection assembly and the compression assembly, the at least one microcontroller configured to selectively dispose the compression assembly into and out of an operative orientation and an inoperative orientation.

2. The wearable garment assembly as recited in claim 1 wherein multi-layer assembly is structured and dimensioned to define a top section corresponding to a shape of the torso and arms of the user.

3. The wearable garment assembly as recited in claim 1 wherein the multilayer assembly is structured and dimensioned to define a bottom section corresponding to a shape of the lower torso and the legs of the user.

4. The wearable garment assembly as recited in claim 1 wherein the at least one microcontroller and the compression structure are collectively configured such that the compression structure exerts a compression force sufficient to at least partially reduce hemorrhaging around at least one limb of the user when the compression assembly is disposed in the operative orientation.

5. The wearable garment assembly as recited in claim 1 wherein the at least one microcontroller and the compression structure are collectively configured such that the compression structure does not exert a compression force on the individual when the compression assembly is disposed in the inoperative orientation.

6. The wearable garment assembly as recited in claim 1 wherein the at least one microcontroller is configured to automatically dispose the compression assembly in the inoperative orientation after about fifteen minutes after it is disposed in the inoperative orientation.

7. The wearable garment assembly as recited in claim 1 wherein the at least one microcontroller and the compression structure are collectively configured to exert a compression force around at least one limb of the user upon at least one of the plurality of sensors detecting a predetermined change in voltage associated with the presence of blood around at least one limb of the user.

8. The wearable garment assembly as recited in claim 7 wherein the compression structure is configured to exert a compression force sufficient to at least partially reduce hemorrhaging around at least one limb of the user upon at least one of the plurality of sensors detecting the predetermined change in voltage associated with the presence of blood around the at least one limb of the user adjacent to the brachial artery, the radial artery, the femoral artery or the popliteal artery.

9. The wearable garment assembly as recited in claim 1 wherein the intended location of the plurality of sensors comprises at least one of the approximate location of the brachial artery, the radial artery, the femoral artery and the popliteal artery of the user.

10. The wearable garment assembly as recited in claim 1 wherein the retention mechanism comprises a plurality of sleeves, the compression structure being at least partially disposed within the plurality of sleeves in non-movable relation thereto.

11. The wearable garment assembly as recited in claim 1 wherein the compression structure comprises at least one of a wire, cable and lace.

12. The wearable garment assembly as recited in claim 1 wherein the compression structure is configured to define a plurality of braids or loops and to provide for an increased coverage around at least one limb of the user.

13. The wearable garment assembly as recited in claim 1 wherein the compression assembly further comprises at least one stool collectively configured with a corresponding segment of the compression structure to wind it on the stool and exert a compression force on a corresponding limb of the user upon a rotation of the stool in a first direction.

14. The wearable garment assembly as recited in claim 13 wherein the at least one stool is collectively configured with the corresponding segment of the compression structure to unwind it from the stool and at least partially reduce the compression force exerted on the corresponding limb of the user upon a rotation of the stool in a second and opposite direction.

15. The wearable garment assembly as recited in claim 14 wherein the compression assembly further comprises a portable motor operatively configured with the stool to selectively induce a rotation of the stool in the first direction and in the second and opposite direction.

16. A wearable garment assembly configured to automatically transmit a signal upon a successful detection of blood within the wearable garment assembly, the wearable garment assembly comprising:
a multi-layer assembly comprising:
a mid-layer disposed between the inner layer and the outer layer, and
a detection assembly comprising a plurality of sensors each one disposed
at an intended location of the inner layer,
a communication assembly,
at least one microprocessor operatively configured with the detection assembly and the communication assembly, the at least one microprocessor and the detection assembly collectively configured to enable the communication assembly to automatically transmit a signal upon at least one of the plurality of sensors detecting a predetermined change in voltage associated with the presence of blood around at least one limb of the user.

17. The wearable garment assembly as recited in claim 16 wherein the communication assembly comprises a transmitter configured to transmit the signal to emergency medical services (EMS).

18. The wearable garment assembly as recited in claim 16 wherein the signal comprises a short message service (SMS) to authorities or first responders.

19. The wearable garment assembly as recited in claim 16 wherein the at least one microprocessor and the detection assembly are collectively configured to enable communication assembly to automatically transmit a signal upon at least one of the plurality of sensors detecting the predetermined change in voltage associated with the presence of blood around the at least one limb of the user adjacent to the brachial artery, the radial artery, the femoral artery or the popliteal artery.

20. A wearable garment assembly configured to exert a pressure on the body of a user upon a successful detection of blood within the garment assembly, the wearable garment assembly comprising:
a multi-layer assembly comprising:
a mid-layer disposed between the inner layer and the outer layer, and
a retention mechanism disposed between the mid-layer and outer layer;
the multi-layer assembly structured and dimensioned to define a top section corresponding to a shape of the torso and arms of the user,
a compression assembly comprising a compression structure disposed within the retention mechanism in slidable relation thereto, and
a detection assembly comprising a plurality of voltage sensors each one disposed at an intended location of the inner layer,
a communication assembly,
at least one microcontroller operatively connected to the detection assembly and the compression assembly, the at least one microcontroller and the detection assembly collectively configured to selectively dispose the compression assembly into and out of an operative orientation,
at least one microprocessor collectively configured with the detection assembly and the communication assembly, the at least one microprocessor and the detection assembly collectively configured to enable the communication assembly to automatically transmit a signal upon the at least one of the plurality of voltage sensors determining a predetermined change in voltage a change in voltage between about 1.22 volts and 1.46 volts and associated with the presence of blood around at least one limb of the user, and
wherein the at least one microcontroller and the compression structure are collectively configured to exert a compression force sufficient to at least partially reduce hemorrhaging around a limb of the user when the compression assembly is disposed in the operative orientation.
